(19) United States
(12) Patent Application Publication
(10) Pub. No.: US 2008/0251105 A1
(43) Pub. Date: Oct. 16, 2008

(54) Cleaning Compositions Comprising Hydrogen Peroxide

(76) Inventors: Christine Toussaint, Aineffe (Faimes) (BE); Nicole Andries, Liege (BE); Genevieve Blandiaux, Trooz (BE); Myriam Mondin, Seraing (BE); Didier Dormal, Aywaille (BE)

Correspondence Address: COLGATE-PALMOLIVE COMPANY, 909 RIVER ROAD, PISCATAWAY, NJ, 08855, US

(21) Appl. No.: 11/735,189

(22) Filed: Apr. 13, 2007

(51) Int. Cl. B08B 3/04; C07C 381/00; C11D 17/04

(52) U.S. Cl. 134/34; 510/438; 510/492

(57) ABSTRACT

The present invention is directed to cleaning compositions and cleaning systems comprising an anionic surfactant, a nonionic surfactant, lactic acid, hydrogen peroxide and water. In various embodiments, the compositions may further comprise an amphoteric or pseudo-cationic surfactant, an alcohol and a glycol ether solvent. The present invention is also directed to cleaning systems comprising the compositions in contained within a bottle having a nozzle and a spray pump dispenser, as well as methods for removing stain and/or soil from a surface.

### BACKGROUND OF THE INVENTION

The present invention is related to cleaning compositions; in particular, those that are useful for removing stains and/or soils from household surfaces, as well as disinfecting, while maintaining good foaming, grease cutting, low residue pattern and superior rinsing and mildness properties.

Home care compositions have long been used for application to household surfaces for cleaning, disinfecting, maintaining hygiene and imparting pleasant odors and other benefits. However, there is an ongoing need for improved compositions exhibiting the ability to clean and disinfect household surfaces such as floors, counters, walls, furniture and fixtures, as well as the ability to provide benefits in laundry applications. Compositions that are stable, mild, easy to rinse, safe, and non-irritating to users and that have antibacterial properties are especially desirable. Particularly desirable are compositions that have superior abilities to remove stains and/or soils while maintaining these desirable properties.

### SUMMARY OF THE INVENTION

The present invention provides, in certain embodiments, a cleaning composition consisting of:

(a) 0.5 to 3.5% by weight of a linear alkyl benzene sulfonate;

(b) 0.5 to 6% by weight of a C₉₋₁₁ alcohol with 8 ethoxylates;

(c) 0.3 to 2% by weight of hydrogen peroxide;

(d) 0.3 to 2% by weight of lactic acid;

(e) water;

(f) optionally cocoamidopropyl betaine;

(g) optionally ethanol,

(h) optionally propylene glycol n-butyl ether;

(i) optionally a dye;

(j) optionally an opacifier;

(k) optionally a thickener;

(l) optionally a fragrance; and

(m) optionally caustic soda;

wherein the composition has a pH of about 3 to about 4.

The present invention is directed, in other embodiments, to a cleaning system comprising a cleaning composition consisting of:

(a) 0.5 to 3.5% by weight of a linear alkyl benzene sulfonate;

(b) 0.5 to 6% by weight of a C₉₋₁₁ alcohol with 8 ethoxylates;

(c) 0.3 to 2% by weight hydrogen peroxide;

(d) 0.3 to 2% by weight lactic acid;

(e) 0.2 to 1% by weight cocoamidopropyl betaine;

(f) 0.5 to 2% by weight ethanol;

(g) 1 to 4% by weight propylene glycol n-butyl ether;

(h) water;

(i) optionally a dye;

(j) optionally an opacifier;

(k) optionally a thickener;

(l) optionally a fragrance; and

(m) optionally caustic soda;

wherein the cleaning composition is contained within a bottle having a nozzle and a spray pump dispenser.

The present invention is directed, in other embodiments, to a cleaning composition consisting of:

(a) 1.5 to 1.75% by weight of a linear alkyl benzene sulfonate;

(b) 3% by weight of a C₉₋₁₁ alcohol with 8 ethoxylates;

(c) 0.75% by weight hydrogen peroxide;

(d) 0.3 to 2% by weight lactic acid;

(e) water;

(f) optionally a dye;

(g) optionally an opacifier;

(h) optionally a thickener;

(i) optionally a fragrance; and

(j) optionally caustic soda;

wherein the composition has a pH of about 3.5.

The present invention is directed, in other embodiments, to a cleaning system comprising a cleaning composition consisting of:

(a) 0.9 to 1.1% by weight of a linear alkyl benzene sulfonate;

(b) 1.25% by weight of a C₉₋₁₁ alcohol with 8 ethoxylates;

(c) 0.75% by weight hydrogen peroxide;

(d) 0.3 to 2% by weight lactic acid;

(e) 0.45% by weight cocoamidopropyl betaine;

(f) 1% by weight ethanol;

(g) 2% by weight propylene glycol n-butyl ether;

(h) water;

(i) optionally a dye;

(j) optionally an opacifier;

(k) optionally a thickener;

(l) optionally a fragrance; and

(m) optionally caustic soda;

wherein the cleaning composition is contained within a bottle having a nozzle and a spray pump dispenser.

The present invention is directed, in certain embodiments, to a cleaning composition consisting of:

(a) 0.5 to 3.5% by weight of a linear alkyl benzene sulfonate;

(b) 0.5 to 6% by weight of a C₉₋₁₁ alcohol with 8 ethoxylates;

(c) 0.3 to 2% by weight hydrogen peroxide;

(d) 0.3 to 2% by weight lactic acid;

(e) 0.01 to 0.3% by weight opacifier;

(f) less than 0.5% by weight dye;

(g) 0.01 to 1.5% by weight fragrance;

(h) 0.01 to 0.5% by weight thickener;

(i) water;

(j) optionally cocoamidopropyl betaine;

(k) optionally ethanol; and

(l) optionally propylene glycol n-butyl ether;

wherein the composition has a pH of about 3.5.

The present invention is directed, in other embodiments, to methods of cleaning or removing a stain or soil from a surface, comprising the steps of applying a composition according to the present invention to a surface; and rinsing or wiping the surface.

### DETAILED DESCRIPTION OF THE INVENTION

As used herein, ranges are a shorthand for describing each and every value within a range, including endpoints. All references cited in the present disclosure are hereby incorporated by reference in their entirety. Where there is a conflict between a definition in the present disclosure and that of a cited reference, the present disclosure controls.

It has been discovered that compositions can be formulated with mixtures of an anionic surfactant, a nonionic surfactant, lactic acid and hydrogen peroxide, resulting in compositions with superior properties as cleaning agents, particularly for household surfaces such as hard surfaces (e.g., tiles, toilets, sinks, fixtures and other bathroom surfaces). Such compositions may be particularly useful in removing mineral deposits or bleachable stains or soils on hard surfaces and fabrics. In certain embodiments, the present invention is directed to cleaning compositions consisting of: an anionic surfactant, a nonionic surfactant, lactic acid, hydrogen peroxide and water. In various embodiments, the compositions may further comprise one or more of the following: an amphoteric or pseudo-cationic surfactant, an alcohol, a glycol ether solvent, a fragrance, a dye, an opacifier, or a thickener.

In certain embodiments, the present invention provides compositions in the form of home care compositions. As used herein, a “home care composition” is any composition that may be useful in cleaning household surfaces. As used herein, a “surface” refers to the surface of any household appliance or fixture, and may include hard surfaces such as counters, sinks, cabinets, walls, the surfaces of appliances such as kitchen appliances (e.g., stoves, conventional or microwave ovens, refrigerators, dishwashers and the like), or bathroom appliances (e.g., sinks, toilets, bathtubs, tiles, shower curtains and doors) wood or glass surfaces, appliances, floors, utensils or dishes, as well as furniture or clothing (including carpets or rugs, cloths, bedding, leather, sponges and mops, polymeric or fabric surfaces or objects made from natural or synthetic materials, e.g., protective gear or sports equipment).

In various embodiments, the present compositions may be in the form of a water-dilutable cleaner or a spray. As used herein, “water-dilutable” refers to a composition that may be (but does not necessarily have to be) diluted with water, for example, in a bucket or other container, prior to use. In certain embodiments, the present invention is directed to a cleaning system that comprises the compositions as described herein, contained within a bottle having a nozzle and a spray pump dispenser.

In various embodiments, the compositions of the present invention are in the form of a microemulsion. The term “microemulsion” may be used to describe, for example, an oil-in-water emulsion, a water-in-oil emulsion or other types of emulsions and microemulsions known in the art.

The compositions of the present invention comprise an anionic surfactant. In certain embodiments, the anionic surfactant is a water soluble linear alkyl benzene sulfonate, such as, for example, the sodium, potassium, ammonium, magnesium and ethanolammonium salts of linear C₈-C₁₆ alkyl benzene sulfonates such as C₁₀-C₁₄ alkyl benzene sulfonates; or the higher alkyl mononuclear aromatic sulfonates, such as the higher alkylbenzene sulfonates containing 9 to 18, or 9 to 16 carbon atoms in the higher alkyl group in a straight or branched chain, or C₈₋₁₅ alkyl toluene sulfonates. Other useful anionic surfactants are discussed in U.S. Pat. No. 6,586,014 to Arvantididou et al. In various embodiments, the anionic surfactant is present in amounts of 0.5 to 3.5%, 0.8 to 2%, 0.9 to 1.1%, 1.6 to 1.7%, 1.62% or 1.65% of the composition.

The compositions of the present invention also comprise a nonionic surfactant. In various embodiments, the nonionic surfactant may be an ethoxylated fatty alcohol, such as a polyalkoxylated fatty alcohol; for example, those described in International Publication WO 2007/001593 to Simon et al. and U.S. Pat. No. 6,342,473 to Kott et al. In certain embodiments, the nonionic surfactant may be a C₉₋₁₁ alcohol with 8 ethoxylates. In various embodiments, the nonionic surfactant is present in amounts of 0.5 to 6%, 1 to 4%, 1.25%, 2 to 3.5% or 3% of the composition.

The compositions of the present invention also comprise hydrogen peroxide. It has been found that the addition of hydrogen peroxide in optimal amounts to provide a composition at optimal pH can improve the ability of the present compositions to remove stains or soils from household surfaces. In various embodiments, the present compositions comprise hydrogen peroxide in amounts of 0.3 to 2%, 0.5 to 1%, 0.6 or 0.75% of the composition.

The compositions of the present invention should be maintained as acidic compositions, that is, having a pH of less than 7. In various embodiments, the pH of the composition should be about 2 to about 5, about 3 to about 4, or about 3.5. The compositions of the present invention also comprise at least one acid such as, for example, lactic acid. In various embodiments, the acid is present in amounts of 0.3 to 2%, 0.4 to 1.75%, 0.45, 0.75, 0.85, 1%, 1.25% or 1.5%. The lactic acid should be present in amounts that will optimize the pH of the overall composition to about 2 to about 5, about 3 to about 4, about 3.2 to about 3.8, or about 3.5.

The compositions of the present invention may further optionally comprise several additional compounds. For example, in certain embodiments, the compositions may further comprise an alcohol. Alcohols that are useful for the present embodiments include monohydric alcohols or polyhydric alcohols (including short chain alcohols), for example, ethanol, ethylene glycol or polyethylene glycol. In certain embodiments, the alcohol is present in the compositions in amounts of 0.5 to 2%, 0.75 to 1.5%, 0.8 to 1.1%, 1% or 1.25% alcohol based on the composition.

Another optional compound that may be useful in the present compositions is an amphoteric or pseuco-cationic surfactant. Examples of suitable amphoteric or pseudo-cationic surfactants include, e.g., amidobetaines, amidosulfobetaines, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, and lauryl bis-(2-hydroxypropyl)alpha-carbox-yethyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine. In various embodiments, the amphoteric or pseudo-cationic surfactant, if present, is present in amounts of 0.2 to 1%, 0.3 to 0.8%, 0.4 to 0.6% or 0.45% of the composition.

Another optional compound that may be useful in the present compositions is a glycol ether solvent. For example, useful solvents include those that have low solubility in water, such as, e.g., propylene glycol n-butyl ether, dipropylene glycol n-butyl ether, dipropylene glycol n-propyl ether, ethylene glycol n-hexyl ether and the like. When present, the glycol ether solvent may be present in amounts of, in various embodiments, 1 to 4%, 1.5 to 3%, 1.75% to 2.75% or 2% of the composition.

The compositions of the present invention are all substantially aqueous. In various embodiments, the compositions comprise at least 75% water, at least 80% water, 85 to 98% water, or 90 to 95% water.

Finally, in various embodiments, the compositions may further comprise additional ingredients, such as, for example, those chosen from the following: colorants (such as pigments or dyes) in amounts of less than 0.5% or 0.00001 to 0.1% or 0.003 to 0.05%, fragrance (such as perfumes) in amounts of 0.01 to 1.5% or 0.1 to 1.25%, or an opacifier in amounts of 0.01 to 0.3% or 0.05 to 0.2%, a thickener in amounts of 0.001 to 1%, 0.01 to 0.5% or 0.05 to 0.2%.

The compositions of the present invention may be made in a variety of ways, including methods among those known in the art for making cleaning and other home care compositions. To maximize the stability of the compositions and maintain their pH in the desired range, caustic soda may be added to adjust the pH prior to incorporation of the hydrogen peroxide in the formulations. The amount of caustic soda added will very depending on the desired pH range. In various embodiments, the amount will be sufficient to maintain the pH in the range of about 2 to about 5, about 3 to about 4, or about 3.5. A skilled artisan can determine the appropriate amount of caustic soda to be added to achieve this goal.

The present invention can be further understood by reference to the following non-limiting Examples.

### EXAMPLE I

Various compositions were prepared with the following components, as set forth in Table 1 below:

TABLE 1
Formulations Tested

| | Formulation 1 | Formulation 2 | Formulation 3 |
|---|---|---|---|
| Ingredient | (BDC) | (BDC) | (Spray) |
| Linear alkyl benzene | 1.65% | 1.62% | 0.99% |
| sulfonate | | | |
| (anionic surfactant) | | | |
| C_{9–11} alcohol with 8 | 3% | 3% | 1.25% |
| ethoxylates (nonionic | | | |
| surfactant) | | | |
| Hydrogen Peroxide | 0.75% | 0.75% | 0.75% |
| Lactic Acid | % | 0.85% | 0.75% |
| Cocoamidopropyl | 0 | 0 | 0.45% |
| betaine | | | |
| Ethanol | 0 | 0 | 1% |
| Propylene Glycol | 0 | 0 | 2% |
| n Butyl Ether | | | |

The tested formulas were able to deliver superior stain and soil removal when applied at full strength to bleachable stains and soils, including tomato, coffee, grass, chocolate and avocado. Performance was compared to that of a standard cleaning formula with no hydrogen peroxide. Specifically, the test was performed as follows: the hard or soft (fabric) surfaces were soiled by depositing thereon the aforementioned staining ingredients. After the depositing step, the surfaces were then allowed to set, and scrubbed and rinsed. The soil removal effectiveness was measured with the Xrite Color Gun, to determine percentage of soil removed from the surface under the test conditions.

Results indicated that stain and soil removal performance was improved by the current compositions (Formulas 1 through 3), when compared to similar compositions not comprising hydrogen peroxide.

We claim:

1. A cleaning composition consisting of:
(a) 0.5 to 3.5% by weight of a linear alkyl benzene sulfonate;
(b) 0.5 to 6% by weight of a C₉₋₁₁ alcohol with 8 ethoxylates;
(c) 0.3 to 2% by weight of hydrogen peroxide;
(d) 0.3 to 2% by weight of lactic acid;
(e) water;
(f) optionally cocoamidopropyl betaine;
(g) optionally ethanol;
(h) optionally propylene glycol n-butyl ether;
(i) optionally a dye;
(j) optionally an opacifier;
(k) optionally a thickener;
(l) optionally a fragrance; and
(m) optionally caustic soda;
wherein the composition has a pH of about 3 to about 4.

2. The composition according to claim 1, wherein the composition has a pH of about 3.5.

3. The composition according to claim 1 in the form of a water-dilutable hard surface cleaning composition.

4. The composition according to claim 1, wherein the cocoamidopropyl betaine is present in an amount of 0.2 to 1% by weight.

5. The composition according to claim 1, wherein the ethanol is present in an amount of 0.5 to 2% by weight.

6. The composition according to claim 1, wherein the propylene glycol n-butyl ether is present in an amount of 1 to 4% by weight.

7. A composition according to claim 1, in the form of a microemulsion.

8. A cleaning system comprising a cleaning composition consisting of:
(a) 0.5 to 3.5% by weight of a linear alkyl benzene sulfonate;
(b) 0.5 to 6% by weight of a C₉₋₁₁ alcohol with 8 ethoxylates;
(c) 0.3 to 2% by weight hydrogen peroxide;
(d) 0.3 to 2% by weight lactic acid;
(e) 0.2 to 1% by weight cocoamidopropyl betaine;
(f) 0.5 to 2% by weight ethanol;
(g) 1 to 4% by weight propylene glycol n-butyl ether;
(h) water;
(i) optionally a dye;
(j) optionally an opacifier;
(k) optionally a thickener;
(l) optionally a fragrance; and
(m) optionally caustic soda;
wherein the cleaning composition is contained within a bottle having a nozzle and a spray pump dispenser.

9. The cleaning system according to claim 8, wherein the cleaning composition has a pH of about 3 to about 4.

10. The cleaning system according to claim 9, wherein the cleaning composition has a pH of about 3.5.

11. A method for cleaning a surface, comprising the steps of applying a composition according to claim 1 to a surface; and rinsing or wiping the surface.

12. A method for removing a stain or soil from a surface, comprising the steps of applying a composition according to claim 1 to a surface; and rinsing or wiping the surface.

13. The method according to claim 11, further comprising the step of diluting the composition with water before applying the composition to the surface.

14. A method for removing a stain or soil from a surface, comprising the steps of dispensing the cleaning composition from the cleaning system according to claim 8; and rinsing or wiping the surface.

15. A cleaning composition consisting of:
(a) 1.5 to 1.75% by weight of a linear alkyl benzene sulfonate;
(b) 3% by weight of a C₉₋₁₁ alcohol with 8 ethoxylates;
(c) 0.75% by weight hydrogen peroxide;
(d) 0.3 to 2% by weight lactic acid;
(e) water;
(f) optionally a dye;
(g) optionally an opacifier;
(h) optionally a thickener;
(i) optionally a fragrance; and
(j) optionally caustic soda;
wherein the composition has a pH of about 3.5.

16. A cleaning system comprising a cleaning composition consisting of:
(a) 1.0 to 1.1% by weight of a linear alkyl benzene sulfonate;
(b) 1.25% by weight of a C₉₋₁₁ alcohol with 8 ethoxylates;
(c) 0.75% by weight hydrogen peroxide;
(d) 0.3 to 2% by weight lactic acid;
(e) 0.45% by weight cocoamidopropyl betaine;
(f) 1% by weight ethanol;
(g) 2% by weight propylene glycol n-butyl ether;
(h) water;
(i) optionally a dye;
(j) optionally an opacifier;
(k) optionally a thickener;
(l) optionally a fragrance; and
(m) optionally caustic soda;
wherein the cleaning composition is contained within a bottle having a nozzle and a spray pump dispenser.

17. The cleaning system according to claim 16, wherein the cleaning system has a pH of about 3.5.

18. A cleaning composition consisting of:
(a) 0.5 to 3.5% by weight of a linear alkyl benzene sulfonate;
(b) 0.5 to 6% by weight of a C₉₋₁₁ alcohol with 8 ethoxylates;
(c) 0.3 to 2% by weight hydrogen peroxide;
(d) 0.3 to 2% by weight lactic acid;
(e) 0.01 to 0.3% by weight opacifier;
(f) less than 0.5% by weight dye;
(g) 0.01 to 1.5% by weight fragrance;
(h) 0.01 to 0.5% by weight thickener;
(i) water;
(j) optionally cocoamidopropyl betaine;
(k) optionally ethanol; and
(l) optionally propylene glycol n-butyl ether; and
(m) optionally caustic soda;
wherein the composition has a pH of about 3.5.

19. The cleaning system comprising the cleaning composition according to claim 18, wherein the cocoamidopropyl betaine is present in an amount of 0.2 to 1% by weight, the ethanol is present in an amount of 0.5 to 2% by weight, and the propylene glycol n-butyl ether is present in an amount of 1 to 4% by weight of the cleaning composition.

20. The cleaning system according to claim 18, wherein the cleaning composition is contained within a bottle having a nozzle and a spray pump dispenser.
